(19) United States
(12) Patent Application Publication
(10) Pub. No.: US 2023/0364335 A1
(43) Pub. Date: Nov. 16, 2023

(54) DEVICE AND METHOD FOR THE METERED DISPENSING OF A LIQUID

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/313,788

(22) Filed: May 8, 2023

(30) Foreign Application Priority Data
May 13, 2022 (EP) 22173185.4

(51) Int. Cl. A61M 5/168; A61M 5/142

(52) U.S. Cl. CPC A61M 5/16804; A61M 5/14276

(57) ABSTRACT

A device and a method for the metered dispensing of a liquid for the purpose of releasing an active substance in a human or animal body, and to a use of a device. A device for the metered delivery of a liquid for the purpose of releasing an active substance in a human or animal body comprises a metering apparatus for metering a defined volume of a liquid, an inlet in fluidic communication with the metering apparatus for supplying liquid into the metering apparatus and an outlet in fluidic communication with the metering apparatus for dispensing the defined volume of the liquid. The metering apparatus comprises a wall which encloses a metering cavity for receiving the liquid. By means of manual pressure on the wall, the metering cavity may be reduced, so that the defined volume of the liquid is delivered from the metering cavity to the outlet.

### CROSS-REFERENCE TO RELATED APPLICATIONS

This application claims priority pursuant to 35 U.S.C. 119(a) to European Application No. 22173185.4, filed May 13, 2022, which application is incorporated herein by reference in its entirety.

### FIELD

The invention relates to a device and a method for the metered dispensing of a liquid for the purpose of releasing an active substance in a human or animal body, and to a use of a device.

### BACKGROUND

Rheumatoid diseases belong to what are known as autoimmune diseases, in which the immune system attacks the body's own structures for reasons that are not yet precisely known. Rheumatoid arthritis and psoriasis arthritis belong to the rheumatic group of diseases and are linked to joint inflammation or arthritis, which are usually chronic and can lead to progressive destruction of the joints. For systemic drug therapy of rheumatoid arthritis and psoriasis arthritis, a number of drug groups are available, for example analgesics, non-steroidal antiphlogistic drugs, glucocorticoids and basic therapeutics (disease modifying anti-rheumatic drugs, dMARD). In addition, there are modern antibodies (biologicals) which block a-TNF (a-tumor necrosis factor) or interleukin IL-17. However, with systemic drug therapy, only relatively small serum concentrations of the active substances can be achieved, since otherwise undesired side effects occur such as damage to the kidneys and liver. Higher serum concentrations are therefore usually avoided in practice.

This has the consequence that the active substance concentrations can only be very low at the destination, e. g., in the tissue of the joint capsule and at tendon insertions, and therefore only a limited immunomodulating effect is possible. To reduce chronic inflammation processes in the joint region of patients with rheumatoid arthritis and psoriasis arthritis and in many other diseases, it would therefore be desirable if the corresponding active substances could be applied locally in the vicinity of the joint capsule and/or affected tendons or tendon insertions, and in particular directly in the affected body structure, in order to achieve a locally high concentration of these active substances. In addition, a large number of additional clinical pictures are known in which only comparatively low concentrations at the target site can be achieved with systemic administration of active substances, even though higher local concentrations would be desirable.

In particular with rheumatic diseases, for pain therapy, for local cytostatic release and for the treatment of tumor diseases, it would also be desirable if the affected patients themselves could apply the active substance solutions as required.

A medical fluid depot which can be implanted under the skin and filled with a cannula is known from the publication EP 3 978 065 A1.

### SUMMARY

The object of the invention is to provide a device, a method and a use by means of which a metered delivery of a liquid for the purpose of releasing an active substance in a human or animal body is possible and which eliminates at least some of the mentioned disadvantages. In particular, a patient should be rendered able to personally release active ingredient if necessary.

The object is achieved by a device according to claim 1 and a method and a use according to the independent claims. Advantageous embodiments are specified in the dependent claims.

In order to achieve the object, a device is used for the metered dispensing of a liquid for the purpose of releasing an active substance in a human or animal body. The device comprises a metering apparatus for metering a defined volume of a liquid, an inlet in fluidic communication with the metering apparatus for supplying liquid into the metering apparatus and an outlet in fluidic communication with the metering apparatus for dispensing the defined volume of the liquid. The metering apparatus comprises a wall which encloses a metering cavity for receiving the liquid. By manually pressing on the wall, the metering cavity may be reduced, such that the defined volume of the liquid is delivered from the metering cavity to the outlet.

The device is capable of metering a defined volume of a liquid through manual pressure and therefore in a simple and reproducible manner. In this way, a simple and safe release of active substance is possible, in particular by the patient themselves. The patient can personally determine the time of delivery of the active substance solution.

The manual pressure can be applied by a finger. The manual pressure is exerted on at least one region of the wall. Active substance solution can be fed through the inlet to the metering apparatus, so that multiple or repeated release of an active substance is possible. The outlet can be arranged in the vicinity of the designated site for the active substance release, in particular in the body of the patient. Likewise, a suitable conduit element can be arranged between the outlet and the designated site. A high active ingredient concentration can therefore be achieved locally without having to accept the known disadvantages of a high serum concentration. Furthermore, due to its simple mechanical mode of operation, the device is simple and robust, cost-effective to manufacture and also safe to use.

In one embodiment, the device is configured to be implanted under the skin (subcutaneously). Alternatively or additionally, the device can be configured such that it can be attached to the skin, for example by adhesion.

The liquid contains in particular at least one active substance. A liquid within the meaning of the invention comprises any type of solution with a liquid solvent.

The metering cavity is a cavity which is used for metering. The metering cavity is reduced by manual pressure. This means that the internal volume of the metering cavity in which liquid may be contained is reduced. As a result, liquid is displaced out of the metering cavity and reaches the outlet. In other words, the metering apparatus is a manually operable mechanical pump. The manual pressure is applied to at least one region of the wall. In particular, the metering cavity can subsequently be enlarged again and refilled with the liquid, so that multiple metering is possible. Liquid can therefore flow through the inlet into the metering cavity and be discharged therefrom in a metered manner through the outlet.

The metering cavity is dimensioned such that it releases the defined volume of the liquid under manual pressure on the wall. It cannot be ruled out that a residual volume remains in the metering cavity. The usable volume therefore does not necessarily correspond to the absolute volume of the metering cavity. By means of the manual pressure, the defined volume of the liquid is pressed in particular to the outlet. In particular, the metering cavity is disk-shaped and/or has a circular shape in a view from above. In particular, the metering cavity has a flat underside and/or a convexly curved upper side. The shape of the metering cavity can accordingly be a plano-convex shape.

The defined volume of the liquid is discharged from the outlet of the device. The delivery can take place directly into the body of the patient. The device can also comprise one or more conduit elements into which the liquid is initially discharged and from which the liquid is then delivered into the body of the patient. A conduit element can be a tube, for example. The delivery is used in particular for the targeted introduction of the liquid into a region of a body of a patient, for example in the vicinity of a joint capsule, a tendon and/or a tendon insertion.

The inlet and outlet are an opening allowing fluidic communication to the metering cavity. A connection option for a conduit element can be present at the inlet and/or at the outlet. The device is suitable for the metered dispensing of a liquid for the purpose of releasing an active substance in a human or animal body. The release of active substance does not necessarily have to take place in the body, but can of course also take place on the particular body.

In one embodiment, at least one region of the wall is designed such that at least the region of the wall is elastically deformed by the manual pressure. In other words, the wall is at least partially elastically deformable. In this way, the metering cavity can be reduced in a particularly simple and reliable manner. In particular, the wall of at least one side of the metering cavity consists at least substantially of an elastically deformable material. In one embodiment, the wall of the entire metering cavity is partially or completely composed of an elastically deformable material. The volume can therefore be easily changed when the filling level of the metering cavity is changed. In one embodiment, the wall of the metering cavity comprises a substantially rigid base plate and an elastic cover element which is mechanically connected to said base plate. A base plate and a cover element are in principle connected to one another in a liquid-tight manner.

In one embodiment, the wall returns at least substantially to its original shape by an elastic restoring force after the manual pressure ceases. An elastic restoring force means the effort of a component or part thereof to move back into the original position or shape. The restoring force is caused in particular by the elasticity of the wall. The wall seeks to return to its original shape. In this way, a negative pressure is generated in the metering cavity. When the external force ceases, the wall can return to its original shape. In particular, liquid flows through the inlet into the metering cavity, preferably sucked in by the arising negative pressure. This embodiment allows the metering cavity to be easily filled and reproducibly metered.

In one embodiment, the metering apparatus comprises a first check valve that prevents flow of the liquid from the metering cavity to the inlet, and/or a second check valve that prevents flow of the liquid from the outlet to the metering cavity. A check valve is a component which allows the flow of a liquid in only one direction. The first check valve is typically designed and arranged such that it only allows the flow of the liquid through the inlet into the metering cavity. The second check valve is typically designed and arranged such that it only allows the flow of the liquid from the metering cavity to the outlet. This allows the metering cavity to be easily filled.

The first check valve can be designed as a ball valve, flap valve and/or as a lip valve. The second check valve can be designed as a ball valve, flap valve and/or as a lip valve. A movable valve body of the valve such as a ball of a ball valve or a flap of a flap valve can have a largest extension or a diameter of at most 1 mm. Valves of this type have proven to be durable and reliable in the present size range, do not react with active substances and are inexpensive to produce. In particular, the check valves can be inserted into cavities of the housing of the device provided for this purpose. In particular, check valves can be locked or screwed therein. For this purpose, check valves in particular have suitable shaped elements for screwing or locking on their outer side.

In one embodiment, the device additionally comprises a supply cavity for receiving a supply of the liquid to be metered. The supply cavity is in fluidic communication with the inlet. A supply cavity is a cavity which is used as a supply. The supply cavity may be connected to the metering cavity or to the inlet via a conduit element.

In one embodiment, the supply cavity is fixedly connected to the inlet and/or to the metering cavity. In this way, a particularly compact device can be provided which comprises both a supply and the metering apparatus. This is advantageous in particular when used as an implant. In one embodiment, the wall of the supply cavity comprises a substantially rigid base plate and an elastic cover element which is mechanically connected thereto. In one embodiment, at least a part of a wall delimiting the supply cavity is produced integrally with at least part of a wall delimiting the metering cavity. For example, a common base plate and/or a common cover element is provided for the supply cavity and the metering cavity. In this way, the device can be produced with little technical effort. For example, the cover element can be glued or welded onto a housing or a base plate. In one embodiment, the metering cavity and the supply cavity are arranged adjacent to each other. Typically, a distance between the metering cavity and the supply cavity and/or the length of a conduit element arranged therebetween is at most 15 mm, in particular at most 10 mm, advantageously at most 5 mm and preferably at most 3 mm.

In particular, a volume of the supply cavity is greater than a volume of the metering cavity at least by a factor of 2, preferably a factor of 5. In this way, multiple metering can be made possible without the supply cavity having to be intermittently filled. In particular, a volume of the supply cavity is greater by a factor of at most 50, typically by a factor of 25 and in one example by a factor of 15 or 10 than the volume of the metering cavity. A supply cavity which by comparison is overly large is associated with an overly large device, which creates practical problems. The different orders of magnitude of the factor result from the very different volumes of different active substances, which volumes are to be metered. Preferably, the metering cavity has a usable volume between 20 μL and 200 μL. However, smaller or larger volumes are also possible as a function of the active ingredient to be metered, the desired dosage quantity and the active ingredient concentration in the liquid. In particular, the supply cavity has a volume greater than 50 μL, in particular 100 μL and preferably 200 μL and/or less than 20 mL, in particular 10 mL and preferably 5 mL. In particular, the details concerning the volume of the supply cavity relate to a usable volume of the supply cavity, which corresponds to the maximum liquid volume of the supply cavity minus any liquid volume remaining in the supply cavity. The supply cavity typically has a maximum extension, for example a maximum diameter of 30 mm.

In particular, the supply cavity is disk-shaped and/or has a circular shape in plan view. In particular, the supply cavity has a flat underside and/or a convexly curved upper side. The shape of the supply cavity can be described as plano-convex.

In one embodiment, the supply cavity is formed by a wall which is elastically deformable at least in some regions, so that the supply cavity decreases as the supply of the liquid decreases. In this way, it is prevented that air or unwanted liquid collects in the supply cavity when the supply of the liquid to be metered decreases. In addition, in this way, the supply cavity can be produced similarly to the metering cavity and optionally together therewith.

In particular, the wall of at least one side of the supply cavity consists at least substantially of an elastically deformable material. In one embodiment, the wall of the entire supply cavity is partially or completely composed of an elastically deformable material. The volume can therefore be easily changed when the filling level of the supply cavity is changed.

In one embodiment, the elastic restoring force of the metering cavity is greater than an elastic restoring force of the supply cavity. In particular, the elastic restoring force of the elastically deformable region of the wall of the metering cavity is greater than the elastic restoring force of the elastically deformable region of the wall of the supply cavity. In this way, it can be ensured that the at least partially reduced volume of the supply cavity is increased again after metering or dispensing the liquid, and liquid is thereby sucked out of the metering cavity. In this way, once metering has been completed, it is particularly easy to pump liquid from the supply cavity into the metering cavity, so that it can then be dosed again. This is done without the need to apply pressure or external force. This is also done when a deformation of the supply cavity or its wall is necessary as a result.

At least a portion of the greater restoring force of the metering cavity compared to the supply cavity can be achieved by different materials. The wall and/or the material of the wall of the metering cavity can have a greater elasticity, for instance a smaller modulus of elasticity, compared to the wall or material of the supply cavity. The material of the wall of the metering cavity can have a greater hardness, for example a greater Shore A hardness, than the material of the wall of the supply cavity. Preferably, the material of the metering cavity has a Shore A hardness greater than 60. The material of the supply cavity preferably has a Shore A hardness of less than 60. In the case of different materials, the wall thicknesses can be the same or different. The mentioned differences can relate to a particular elastic part of the wall of the particular cavity or to the entire wall.

At least a portion of the greater restoring force of the metering cavity compared to the supply cavity can be achieved by different wall thicknesses. The wall thickness of the metering cavity may be greater than the wall thickness of the supply cavity. In the case of different wall thicknesses, the materials can be the same or different.

In one embodiment, at least one region of the wall of the supply cavity is designed such that a cannula (injection cannula) can pierce through at least the region of the wall of the supply cavity, in order to introduce liquid into the supply cavity with the cannula.

In other words, at least one region of the wall is designed as a piercing region. In particular, the wall of at least one side of the supply cavity, in particular of the entire supply cavity, is produced partially or completely from a material that can be pierced by a cannula. An elastic region of the wall, for example an elastic cover element, is preferably designed as a piercing region. In this way, the supply can be filled in a simple manner and without complicated opening of the cavity. This is particularly advantageous when used as an implant, wherein the cannula is guided through the skin layer and optionally fabric layer covering the implant. For example, medical personnel or the patient themselves can fill the supply of liquid by means of a conventional injection cannula or with a suitable pen. The device can therefore be used on a long-term basis without any explanation or removal of the device from the skin surface being necessary. When the injection cannula is withdrawn, the produced perforation can close off the wall, in particular as a result of the restoring force of the elastically deformable material.

In one embodiment, the device has a nonpierceable element to prevent undesired piercing of a rear region of the wall. The rear region is the region opposite to the region of the wall that is to be pierced, or the rear region of the supply cavity. In particular, the nonpierceable element is arranged on the rear region of the wall. This means in particular the wall of the supply cavity. For example, the nonpierceable element is arranged on a base plate of the device. The nonpierceable element cannot be pierced by manually operated injection cannulas or with a pen. It stops the movement of the injection cannula. This reliably prevents penetration of the base of the supply cavity, injury to the tissue lying under the base and damage to the device.

In one embodiment, the nonpierceable element is a nonpierceable plate. This allows protection with very little effort against unwanted piercing. In one embodiment, the nonpierceable element is produced from a biocompatible material. In this way, interactions with the body of the patient and with the liquid and active substances contained therein are prevented. The nonpierceable element is preferably made of metal. In particular, the nonpierceable element comprises titanium, titanium alloys, tantalum, tantalum alloys, silver, silver alloys and/or stainless steel, or consists of one or more of the cited materials.

In one embodiment, the nonpierceable element is arranged at least partially in the supply cavity. “At least partially in the supply cavity” means that at least a portion of a surface of the nonpierceable element is in contact with the liquid located in the supply cavity. This makes manufacturing very easy. In one embodiment, at least a portion of the surface of the nonpierceable element is coated with silver. The silver can have an antiseptic effect by releasing silver ions. The liquid located in the supply cavity can therefore be protected against microbial contamination.

In one embodiment, the device comprises one or more conduit elements, which are connected or can be connected to the outlet, of an elastically deformable material, in particular plastics material. One or more conduit elements can be provided as a tube. Each conduit element has in particular an outer diameter ≤3 mm. At least one conduit element is in particular implantable, in order to be positioned in the body as close as possible to the intended release location.

In one embodiment, one or more of the conduit elements comprises perforations spaced apart from each other, wherein the perforations are arranged in particular along the longitudinal axis. When pressure is applied, each perforation is opened by a liquid located in the conduit element, so that the pressurized liquid exits. Each perforation automatically closes after the end of the application of pressure, in particular by the restoring force of the elastic material. The conduit elements are preferably designed, in particular perforated, according to the teaching of the unexamined patent application EP3795196A1 and/or EP3795196A1. In an additional embodiment, a valve is arranged at the end of one or more conduit elements, which valve is reversibly opened when pressure is applied by the liquid, and which closes again after the end of the application of pressure. In both mentioned embodiments, no permanent openings are present on the conduit elements. In this way, clogging, for example from ingrowth of connective tissue or through penetration of blood, is prevented. The conduit elements can therefore be used over a period of weeks up to several months.

In one embodiment, the device has a maximum height of less than 13 mm, in particular less than 10 mm. The height relates to the total height of a main body of the device which in particular comprises the metering apparatus, the inlet, the outlet and optionally the supply cavity. Any conduit elements, such as tubes connected to the outlet, are not taken into account.

Similarly, the width and the length refer to particular overall dimensions of the main body. The maximum height is the height at the highest position. This applies analogously to the width and length. The highest position relates to a state of the device in which the supply cavity and/or the metering cavity is filled completely or as intended.

In other words, the device is flat. In this way, the device can be implanted particularly easily. The maximum height is preferably less than 7 mm. The maximum height is typically greater than 4 mm. In one embodiment, the device has a substantially flat underside. This can be provided by a substantially flat base plate. The base plate can be substantially stiff. In this way, the device can be implanted particularly easily. In particular, the device is disk-shaped.

In one embodiment, a width of the device is greater than a height of the device at least by a factor of 3. In particular, the factor is greater than 4 and/or less than 10, preferably less than 6.

In each case, a maximum width and a maximum height are meant. In one embodiment, a length of the device is greater by at least a factor of 5 than a height of the device. In particular, the factor is greater than 7 and/or less than 15, preferably less than 10. In each case, a maximum length and a maximum height are meant.

In one embodiment, the wall of the metering cavity is convex at least in some regions. The convex region is preferably the region of the wall to be pressed manually for pumping. In this way, the point to be pressed can be easily felt. This is advantageous in particular when the device is used as an implant, since it can be felt through the skin in this case.

During implantation, the device and optionally the one or more conduit elements are arranged under the skin. In particular, the device filled with the liquid is implanted. This prevents air in the device that could be released during the first metering. The conduit elements are typically positioned such that active ingredients can take be released at or near a designated site. For example, the device is implanted on a back of a hand, in order to be able to feed liquid in a targeted manner to rheumatic finger joints.

Another aspect of the invention is a method for the metered dispensing of a liquid for the purpose of releasing an active substance in a human or animal body using a device according to the invention. By manually pressing on the wall, the metering cavity is reduced, so that a defined volume of the liquid is delivered from the metering cavity to the outlet. All features, advantages and effects of the device mentioned at the outset also apply to the method and vice versa. In one embodiment, the device is implanted under the skin in a human or animal body. In an alternative embodiment, the device is fastened, in particular glued, to the skin surface. In one embodiment, the method is a therapeutic or a non-therapeutic method. The liquid may contain as an active ingredient one or more of the group comprising analgesics, non-steroidal antiphlogistic drugs, glucocorticoids, basic therapeutics (disease modifying anti-rheumatic drugs, dMARD), biologicals and immune modulators, preferably those which are hydrolytically stable over a prolonged period.

An additional aspect of the invention is a method for filling the device according to the invention. A cannula pierces a region of the wall of the supply cavity, in order to introduce liquid into the supply cavity with the cannula. All features, advantages and effects of the above-mentioned aspects also apply to the method and vice versa.

Another aspect of the invention is the use of a device according to the invention for the metered dispensing of a liquid for the purpose of releasing an active substance in a human or animal body. In particular, by manually pressing on the wall, the metering cavity is reduced, so that a defined volume of the liquid is delivered from the metering cavity to the outlet. In particular, the device is implanted in a human or animal body under the skin, or is attached, in particular glued, to the skin surface. In one embodiment, the use is a therapeutic or non-therapeutic use. All features, advantages and effects of the above-mentioned aspects also apply to the use and vice versa.

### BRIEF DESCRIPTION

Exemplary embodiments of the invention are also explained in more detail below with reference to figures. Features of the exemplary embodiments can be combined individually or in a plurality with the claimed objects, unless otherwise indicated. The claimed areas of protection are not limited to the exemplary embodiments.

Shown are:

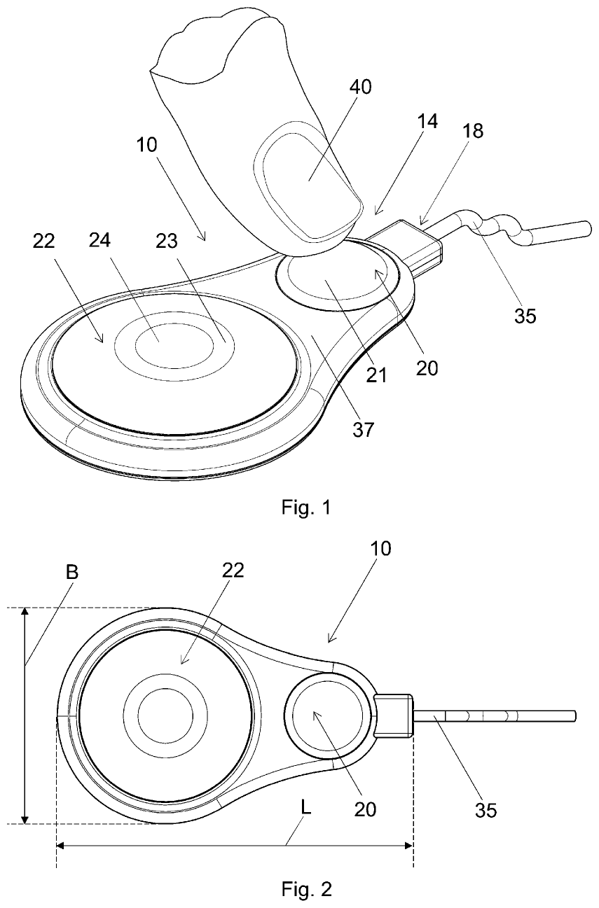

FIG. 1: a perspective view of a device according to the invention,

FIG. 2: a plan view of a device according to the invention,

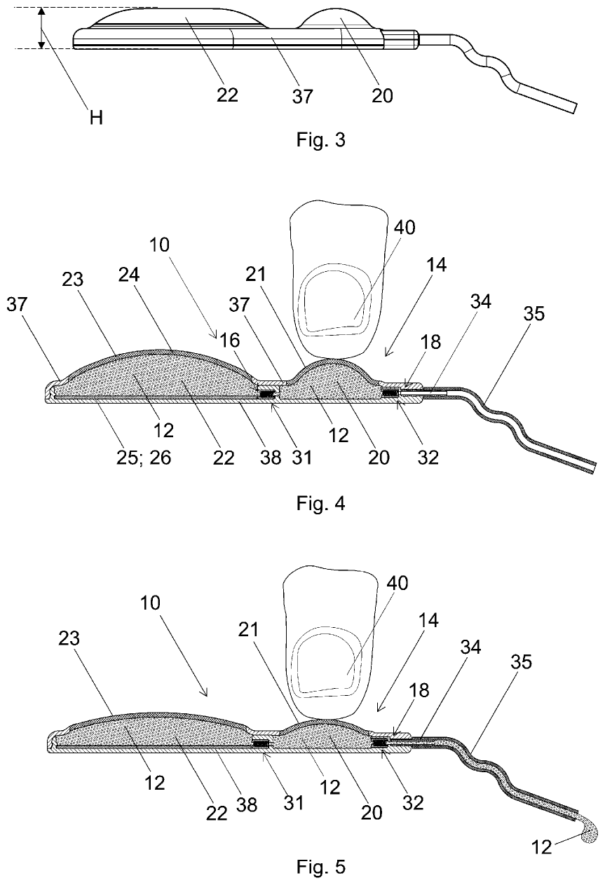

FIG. 3: a side view of a device according to the invention;

FIG. 4: a sectional drawing of a device according to the invention while performing a method step,

FIG. 5: a sectional drawing of a device according to the invention while performing an additional method step;

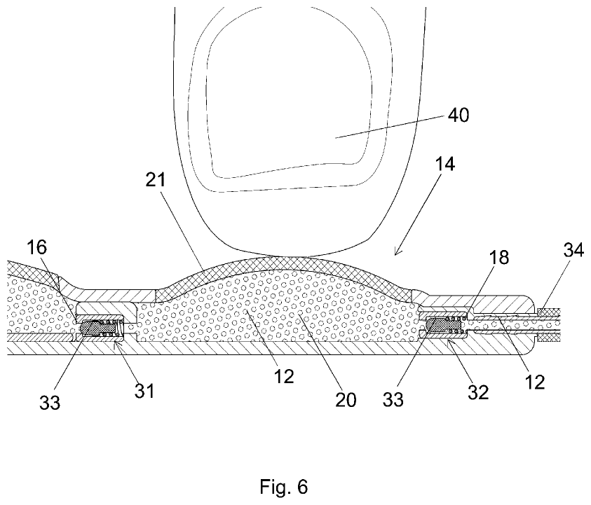

FIG. 6 an enlarged detail from FIG. 5,

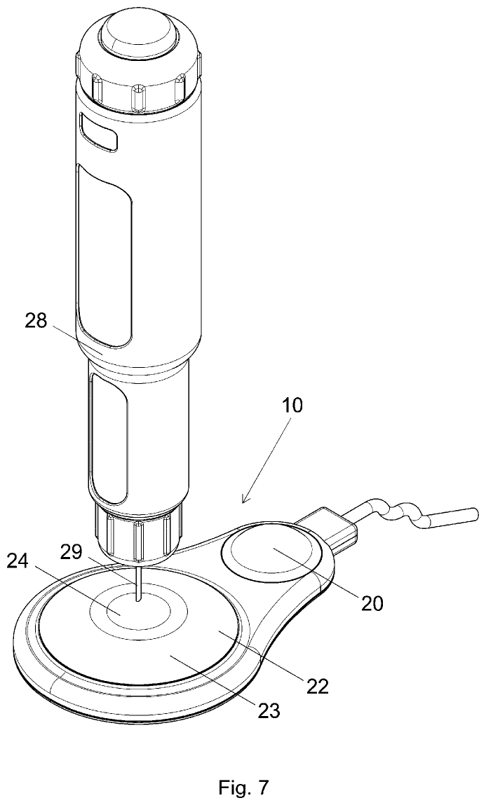

FIG. 7 a perspective view of a device according to the invention,

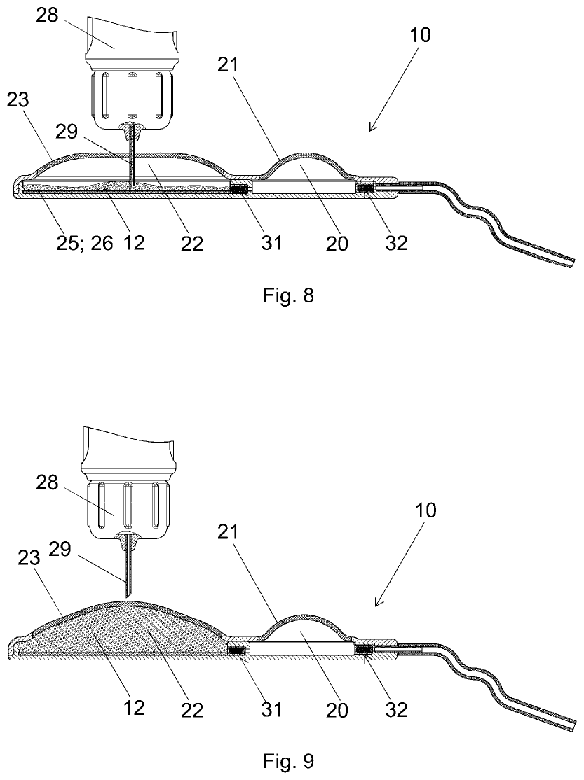

FIG. 8 a sectional drawing of a device according to the invention while filling, and

FIG. 9 a sectional drawing of a device according to the invention after filling.

### DETAILED DESCRIPTION

FIG. 1 shows a device 10 for the metered dispensing of a liquid for the purpose of releasing an active substance in a human or animal body. The device 10 comprises a housing 37 in which a metering apparatus 14 is accommodated. The metering apparatus 14 comprises a metering cavity 20, which is delimited at the top by an elastic wall 21. By manually pressing with a finger 40 on the wall 21, a defined volume of a liquid from the metering cavity 20 can be output from an outlet 18 of the device. A conduit element, namely a tube 35, through which the metered volume can be guided to a target position, is connected to the outlet 18.

As an inlet, the metering cavity 20 comprises a fluidic connection to a supply cavity 22, which is likewise accommodated in the housing 37. The supply cavity 22 is also delimited upward by an elastic wall 23. After the liquid has been metered, liquid can therefore flow out of the supply cavity 22 into the metering cavity 20, in order to allow renewed metering.

FIG. 2 shows a plan view, and FIG. 3 shows a side view. It can be seen that both the width B and the length L of the device 10 are significantly larger than the height H of the device 10. The width B of the device 10 is greater by a factor of between 5 and 6 than the height H of the device. The length L of the device 10 is greater by a factor between 8 and 9 than the height H of the device. The height H of the device is between 5 mm and 7 mm. Overall, the device 10 is flat, plate-shaped and compact, in order to allow implantation under the skin of a patient. The usable volume of the supply cavity 22 is, in particular, greater by a factor between 6 and 10 than the usable volume of the metering cavity 20, so that 6 to 10 dosage operations are possible.

FIGS. 4 and 5 show a possible layout and use of the device 10 in a longitudinal section. The metering apparatus 14 with the metering cavity 20 and inlet 16 and outlet 18 are shown. The supply cavity 22 is connected to the inlet 16. A tube connection 34 with a tube 35 connected thereto is located at the outlet 18. A first check valve 31 allows a flow of liquid from the supply cavity 22 through the inlet 16 into the metering cavity 20, but not in the opposite direction. A second check valve 32 allows a flow of liquid from the metering cavity 20 to the outlet 18, but not in the opposite direction. This ensures that the direction of flow of the liquid is always as intended. Penetration of metered liquid or bodily fluid into the device is prevented.

The housing 37 of the device comprises a base plate 38 and outer and central upper housing parts. The housing 37 is produced from a comparatively stiff plastics material. The upper regions of the wall 21 of the metering cavity 20 and the wall 23 of the supply cavity 22, which are fastened to the housing 37, are produced from an elastic material and are accordingly deformable.

The elastic wall 23 of the supply cavity 22 is designed such that it can be pierced by a cannula, in order to introduce liquid 12 into the supply cavity 22. This is shown in FIGS. 7 to 9, which will be discussed below. The device 10 additionally comprises a nonpierceable element 25, which prevents undesired piercing of the base plate 38. The nonpierceable element 25 is formed as a nonpierceable plate 26 made of a biocompatible metal material, which is arranged on the upper side of the base plate 38 in the interior of the supply cavity 22. The device can therefore be shown in an X-ray. The upper side of the nonpierceable plate 26 is preferably coated with silver.

FIG. 4 shows a state in which both the metering cavity 20 and the supply cavity 22 are filled with the liquid 12. The particular flexible walls 21 and 23 are each curved convexly upward. This makes it possible to very easily feel the metering cavity 20 and therefore the point at which the manual pressure is to be applied.

FIG. 5 shows a subsequent state in which liquid 12 is discharged through the outlet 18, the tube connection 34 and the tube 35 by manually pressing a finger 40 onto the wall 21 of the metering cavity 20. The first check valve 31 is blocked, so that no liquid can flow from the metering cavity 20 into the supply cavity 22. By contrast, the second check valve 32 is open, so that the liquid can reach the outlet 18 from the metering cavity 20.

On the one hand, the convex curvature of the metering cavity 20 and therefore its volume is reduced in FIG. 5 in contrast to FIG. 4. This can be additionally continued by further pressing down a finger 40 until an end point is reached, for example when the wall 21 touches the base plate 38 at least in the middle. In other words, the metering cavity 20 is reduced to convey the defined volume of the liquid 12 from the metering cavity 20 to the outlet 18. In this case, the defined volume of the liquid 12 was then metered. On the other hand, the convex curvature of the supply cavity 22 and therefore its volume is also reduced. This can be the case, for example, because one or more dosing operations have already been carried out beforehand.

After metering has taken place, the finger 40 can be removed. Due to the elastic restoring force of the wall 21, a negative pressure is generated in the metering cavity 20, which causes the first check valve 31 to be opened. In this way, liquid 12 can flow out of the supply cavity 22 into the metering cavity 20. In so doing, the wall 23 of the supply cavity 22 can be deformed.

FIG. 6 shows an enlarged detail of FIG. 5. It can be seen that the valve body 33 of the first check valve is in its closed position. It is pressed to the left against a valve housing by the force of a compression spring arranged on the right in the valve and, if applicable, additionally by the pressure of the liquid 12 inside the metering cavity 20, and therefore blocks the flow through the valve. In contrast, the valve body 33 of the second check valve 32 is in an open position. As a result of the pressure of the liquid 12 in the interior of the metering cavity 20, the valve body 33 is pressed to the right against the force of the pressure spring arranged on the right in the valve, so that a gap is produced on the left side between the valve body 33 and the valve housing, through which gap the liquid 12 can flow past the valve body 33 and out through the outlet 18.

FIG. 7 shows the device 10 according to the invention before filling the supply cavity 22 with liquid. A pen 28 with a cannula 29 is arranged directly above the pierceable region 24 of the wall 23 of the supply cavity 22. FIG. 8 shows that the cannula has pierced through the wall 23 into the supply cavity 22. The nonpierceable plate 26 prevents undesired piercing of the region opposite the wall 23, in this case the base plate 38. It is shown that liquid 12 flows out of the pen 28 through the cannula 29 into the supply cavity 22. Due to the restoring force of the spring in the first check valve 31, said valve remains closed, and the liquid does not flow into the metering cavity 20.

FIG. 8 shows a subsequent step in which the supply cavity 22 is completely filled, and the pen 28 with the cannula 29 has already been pulled out of the wall 23. The wall 23 of the supply cavity 22 is curved slightly upward. As an alternative to the illustration shown here, filling can also occur in such a way that the metering cavity 20 is also filled with the liquid 12. For this purpose, the liquid 12 must exert a pressure in the supply cavity 22 such that the first check valve 31 opens. Preferably, the spring force in the second check valve 32 is then larger, so that an unplanned outflow of the liquid through the second check valve 32 is prevented.

### LIST OF REFERENCE SIGNS

Device 10
Liquid 12
Metering apparatus 14
Inlet 16
Outlet 18
Metering cavity 20
Wall 21
Supply cavity 22
Wall 23
Region 24
Nonpierceable element 25
Nonpierceable plate 26
Pen 28
Cannula 29
First check valve 31
Second check valve 32
Valve body 33
Height H
Width B
Length L
Tube connection 34
Tube 35
Housing 37
Floor plate 38
Finger 40

What is claimed is:

1. A device for the metered delivery of a liquid for the purpose of releasing an active substance in a human or animal body, comprising a metering apparatus for metering a defined volume of a liquid, an inlet in fluidic communication with the metering apparatus for supplying liquid to the metering apparatus and an outlet in fluidic communication with the metering apparatus for delivering the defined volume of the liquid, wherein the metering apparatus comprises a wall which encloses a metering cavity for receiving the liquid, wherein the metering cavity can be reduced by manual pressure on the wall, such that the defined volume of the liquid is conveyed from the metering cavity to the outlet.

2. The device according to claim 1, wherein the wall is designed such that at least one region of the wall is elastically deformed by the manual pressure and the wall returns at least substantially to its original shape by an elastic restoring force after the manual pressure ceases.

3. The device according to claim 1, wherein the metering apparatus comprises a first check valve that prevents flow of the liquid from the metering cavity to the inlet, and a second check valve that prevents flow of the liquid from the outlet to the metering cavity.

4. The device according to claim 1, wherein the device additionally comprises a supply cavity for receiving a supply of the liquid to be metered, wherein the supply cavity is in fluidic communication with the inlet, wherein a volume of the supply cavity is greater, in particular at least by a factor of 2, preferably a factor of 5, than a volume of the metering cavity.

5. The device according to claim 1, wherein the supply cavity is formed by a wall which is elastically deformable at least in some regions, so that the supply cavity decreases as the supply of the liquid decreases.

6. The device according to claim 5, wherein the elastic restoring force of the metering cavity is greater than an elastic restoring force of the supply cavity.

7. The device according to claim 4, wherein at least one region of the wall of the supply cavity is designed such that a cannula can pierce through the wall of the supply cavity, in order to introduce liquid into the supply cavity with the cannula.

8. The device according to claim 1, wherein the device has a nonpierceable element, so that undesired piercing of a rear region of the wall of the supply cavity is prevented.

9. The device according to claim 1, wherein the nonpierceable element is a nonpierceable plate (26) made of a biocompatible material, which is arranged at least partially in the supply cavity.

10. The device according to claim 1, wherein the device has a maximum height (H) less than 13 mm, in particular less than 10 mm.

11. The device according to claim 1, wherein a width (B) of the device is greater by at least a factor of 3 than a height (H) of the device and/or in that a length (L) of the device is greater by at least a factor of 5 than a height (H) of the device.

12. The device according to claim 1, wherein the wall of the metering cavity is convex at least in some regions.

13. A method for metered delivery of a liquid for the purpose of releasing an active substance in a human or animal body using a device according to claim 1, wherein by manual pressure on the wall, the metering cavity is reduced so that a defined volume of the liquid is delivered from the metering cavity to the outlet.

14. The method according to claim 1, wherein the device is implanted under the skin in a human or animal body.

15. A use of a device according to claim 1 for the metered delivery of a liquid for the purpose of releasing an active substance in a human or animal body, wherein the device is implanted under the skin, in particular in a human or animal body.
